(19) United States
(12) Patent Application Publication
(10) Pub. No.: US 2009/0062245 A1
(43) Pub. Date: Mar. 5, 2009

(54) Modulation of Neurodegenerative Diseases Through The Progesterone Receptor

(75) Inventors: Sean Scott, San Francisco, CA (US); Daniel Benjamin, Englishtown, NJ (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ALSGEN, INC., Monmouth Junction, NJ (US)

(21) Appl. No.: 12/255,168

(22) Filed: Oct. 21, 2008

Related U.S. Application Data
(63) Continuation of application No. 11/365,962, filed on Mar. 1, 2006.
(60) Provisional application No. 60/658,630, filed on Mar. 4, 2005.

(51) Int. Cl. A61K 31/57

(52) U.S. Cl. 514/170; 514/177; 514/178

(57) ABSTRACT

Methods for modulating hormonal pathways involving the progesterone receptor in a subject with a neurodegenerative disorder, are provided. Progesterone receptor activity is modulated by administering an effective amount of an progesterone receptor modulating pharmacological agent to a subject such that the progesterone receptor modulating pharmacological agent interacts with the progesterone receptor and alters the expression of a protein associated with the neurodegenerative disease.

### RELATED APPLICATION

This application is a continuation of U.S. application Ser. No. 11/365,962 filed Mar. 1, 2006, entitled “Modulation of Neurodegenerative Diseases Through the Progesterone Receptor,” which claims the benefit of priority to U.S. Provisional Application No. 60/658,630, filed Mar. 4, 2005, entitled “Modulation of Neurodegenerative Diseases through the Progesterone Receptor,” the contents of which are hereby incorporated by reference in their entirety.

### BACKGROUND OF THE INVENTION

Amyotrophic lateral sclerosis (ALS) is the most commonly diagnosed progressive motor neuron disease. The disease is characterized by degeneration of motor neurons in the cortex, brainstem and spinal cord (Principles of Internal Medicine, 1991 McGraw-Hill, Inc., New York; Tandan et al. (1985) Ann. Neurol, 18:271-280, 419-431). The cause of the disease is unknown and ALS may only be diagnosed when the patient begins to experience asymmetric limb weakness and fatigue, localized fasciculation in the upper limbs and/or spasticity in the legs which typifies onset. There is a genetic component to at least some incidences of ALS.

In almost all instances, sporadic ALS and autosomal dominant familial ALS (FALS) are clinically similar (Mulder et al. (1986) Neurology, 36:511-517). It has been shown that in some but not all FALS pedigrees the disease is linked to a genetic defect on chromosome 21q (Siddique et al., (1991) New Engl. J. Med., 324:1381-1384).

In particular, mutations in the SOD-1 gene which is localized on chromosome 21q, appear to be associated with the familial form of ALS. The deleterious effects of various mutations on SOD-1 are most likely mediated through a gain of toxic function rather than a loss of SOD-1 activity (Al-Chalabi and Leigh, (2000) Curr. Opin. Neurol., 13, 397-405; Alisky et al. (2000) Hum. Gene Ther., 11, 2315-2329). While the toxicity is unclear, there exists evidence to suggest that elimination of the protein itself will ameliorate the toxicity.

A need exists to develop therapies that can alter the course of neurodegenerative diseases or prolong the survival time of patients with such diseases. In particular, a need exists to reduce the SOD-1 protein produced in the brain and spinal cord of ALS patients. Preventing the formation of wild type or mutant SOD-1 protein may stop disease progression and allow for amelioration of ALS symptoms.

### SUMMARY OF THE INVENTION

Methods and compositions for treatment of neurodegenerative diseases by modulating the activity of a progesterone receptor within neural cells are disclosed. The progesterone receptor is a ligand activated transcription factor that binds progesterone and its analogues with high affinity and acts directly on genomic DNA to inhibit or activate the expression of a broad spectrum of genes. The progesterone receptor is found in the neural cell, e.g., neuronal cells of the spinal cord and nearly all such cells in both males and females, and thus constitutes a useful therapeutic target for treating neurodegenerative diseases, e.g., ALS.

The methods and compositions of the invention can be used to reduce or inhibit the expression of a protein associated with a neurodegenerative disease, e.g., SOD-1 by administering a progesterone related compound e.g., norethindrone, which acts through the progesterone receptor to inhibit SOD-1 mRNA transcription or the stability of the transcript. The decreases in SOD-1 mRNA then leads to decreased protein levels of SOD-1, which reduce its accumulation in the cell and ameliorate the disease. The expression and accumulation of mutant SOD-1 is a widely accepted pathophysiological mechanism underlying familial ALS, and might also play a role in the sporadic form of the disease.

Accordingly, in one aspect, the invention pertains to a method for reducing the production of an SOD protein in a cell comprising, administering a progesterone receptor modulating pharmacological agent to the cell, such that the agent interacts with a progesterone receptor and inhibits transcription of a gene encoding the SOD protein. The cell can be a neural cell, or any cell in the spinal cord, the meningial tissue, or a muscle cell, for example in a subject with ALS (e.g., familial ALS). The SOD protein can be the SOD-1 protein. Examples of cells include, but are not limited to neurons, interneurons, glial cells, microglial cells, muscle cells, cells involved in the immune response and the like.

The progesterone receptor modulating pharmacological agent can be selected from the group consisting of levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, or (17-deacetyl) norgestimate, and analogs thereof. In one embodiment, the progesterone receptor modulating pharmacological agent is progesterone and analogs thereof. In another embodiment, the progesterone receptor modulating pharmacological agent is norethindrone and analogs thereof.

The inhibition of transcription of the gene comprises monitoring by measuring the expression levels of the SOD protein, e.g., the SOD-1 protein. Alternatively, the inhibition of transcription of the gene comprises monitoring the levels of a nucleic acid molecule that encodes the SOD protein, for example by monitoring the ribonucleic acid or deoxynucleic acid levels.

In another aspect, the invention pertains to a method for preventing, ameliorating or treating the symptoms or progression of ALS in a subject by administering a therapeutically effective amount of a progesterone receptor modulating pharmacological agent to the subject, wherein the agent interacts with an progesterone receptor and inhibits transcription of a gene encoding a SOD-1 protein. The ameliorating of symptoms can be monitored by measuring the survival prolongation of the subject, for example by monitoring a neurological score of the subject. alternatively, the amelioration can be determined by monitoring the expression levels of the SOD-1 protein or the levels of a nucleic acid molecule that encodes SOD-1 protein.

### BRIEF DESCRIPTION OF DRAWINGS

FIG. 1 is a graph showing the reduction of SOD-1 protein expression by norethindrone.

FIG. 2 is a bar graph showing the dose related reversal the effect of norethindrone with the progesterone antagonist, mifepristone (RU-486).

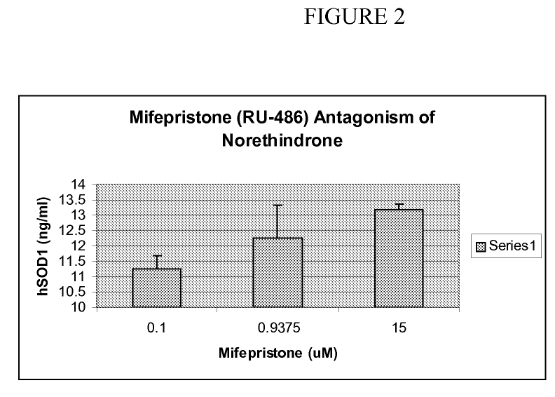

FIG. 3 is a bar graph showing reduced expression of mRNA for alpha synuclein in HeLa cells following treatment with pyrimethamine (ALG-2001) and norethindrone (ALG-3001).

FIG. 4 is a bar graph showing reduced SOD-1 protein in mouse spinal cord following 14 days of intraperitoneal (ip) norethindrone treatment in G93A mice.

### DETAILED DESCRIPTION

The practice of the present invention employs, unless otherwise indicated, conventional methods of microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, Vol. I & II (B. N. Fields and D. M. Knipe, eds.)).

So that the invention is more clearly understood, the following terms are defined:

The term “neurodegenerative disorder” or “neurodegenerative disease” are used interchangeably herein and refer to an impairment or absence of a normal neurological function, or presence of an abnormal neurological function in a subject, or group of subjects. For example, neurological disorders can be the result of disease, injury, and/or aging. As used herein, neurodegenerative disorder also includes neurodegeneration which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, Amyotrophic Lateral Sclerosis (ALS), multiple sclerosis, Huntington's disease, Parkinson's disease, Alzheimer's disease, prion associated disease (CJD), spinal muscular atrophy, spinal cerebellar ataxia, and spinal cord injury.

The terms “modulate” or “modulating” or “modulated” are used interchangeable herein also refer to a change SOD-1 activity, or the expression, i.e., an increase or decrease in SOD-1 activity, or expression, such that the modulation produces a therapeutic effect in a subject, or group of subjects. A therapeutic effect is one that results in an amelioration in the symptoms, or progression of ALS. The change in activity can be measured by quantitative or qualitative measurements of the SOD-1 protein level for example by Western blot analysis. The quantitative assay can be used to measure downregulation or upregulation of SOD-1 protein levels in the presence of a progesterone receptor modulating agent, such as norethindrone. A suitable progesterone receptor modulating agent can be one that down-regulates SOD-1 expression by about 5 percent to about 50 percent compared with a control. The change in expression can also be measured by quantitative or qualitative measurements of the nucleic acid level associated with SOD-1, for example by measuring the expression level of RNA or DNA.

The effect of progesterone receptor modulation on a subject, or group of subjects, can also be investigated by examining the survival of the subject, or group of subjects. For example, by measuring the change in the survival, or the prolongation of survival in one or more animal models for a neurodegenerative disease, e.g., ALS. The change in the survival can be due to the administration of a progesterone receptor modulator agent such as norethindrone that is administered to an ALS murine model. The effect of the progesterone receptor pharmacological modulating agent on the progesterone receptor can be determined based on the increase in days of survival of a test group of ALS mice compared with a control group of ALS mice that have been given a control agent, or no agent. In one embodiment, the progesterone receptor modulating agent increases the percentage effect on survival of the subject, or a population of subjects (e.g., a male population, or a female population) by at least 2% to about 100%. Preferably the percentage effect on survival of the subject, or a population of subjects, is by at least 5% to about 50%, by at least 10% to about 25%. Even more preferably, the percentage effect on survival of the subject, or a population of subjects, is by at least 3%, 4%, 5%, 6%, 7%, 8%, 9%, 10%, 12%, 14%, 16%, 18%, 20%, 22%, 24%, 26% 28%, 30%, 32%, 34%, 36%, 38%, 40%, 42%, 44%, 46%, 48% and 50%. The effect of progesterone receptor modulation may also determined by examining the neurological score of a subject, or group of subjects for example, by assessing the improvement in muscular movement, or by examining the alleviation or amelioration of the disease symptoms. In a preferred embodiment, the neurological score of a subject, or group of subjects is significantly different from that of the untreated control subjects, with a level of significance between p<0.05 and p<0.0001, as determined using standard statistical analysis procedures.

The terms may also be used to refer to a change in the progesterone receptor activity, structure, or the expression of a progesterone receptor, or a subunit of the progesterone receptor, i.e., an increase or decrease in progesterone receptor activity, or expression, such that the modulation produces a therapeutic effect in a subject, or group of subjects.

The terms “pharmacological agent” and “progesterone receptor modulating pharmacological agent” as used herein, are intended to be used interchangeably, and these terms refer to the compound, or compounds, that are used to modulate the progesterone receptor activity in a subject. Preferably, the progesterone receptor modulating pharmacological agent is progesterone, for example, norethindrone. The terms “pharmacological agent” or “progesterone receptor modulating pharmacological agent” are also intended to include other compounds with a similar structure and function to progesterone.

The term “inhibit” or “inhibiting” as used herein refers to a measurable reduction of expression of a target gene or a target protein, e.g., SOD-1. The term also refers to a measurable reduction in the activity of a target protein. Preferably a reduction in expression is at least about 10%. More preferably the reduction of expression is about 20%, 30%, 40%, 50%, 60%, 80%, 90% and even more preferably, about 100%.

The phrase “a disorder associated with SOD activity” or “a disease associated with SOD activity” as used herein refers to any disease state associated with the expression of SOD protein (e.g., SOD-1, SOD-2, SOD-3, and the like). In particular, this phrase refers to the gain of toxic function associated with SOD protein production. The SOD protein can be a wild type SOD protein or a mutant SOD protein and can be derived from a wild type SOD gene or an SOD gene with at least one mutation.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

### I. Neurodegenerative Diseases

In one aspect, the invention pertains to altering the expression of an SOD protein in a cell by administering an progesterone receptor modulating pharmacological agent. The cell can be a neural cell associated in a neurodegenerative disease that involves an SOD protein, such as amyotrophic lateral sclerosis (ALS). The progesterone receptor is a ligand activated transcription factor that binds progesterone and its analogues with high affinity and acts directly on genomic DNA to inhibit or activate the expression of a broad spectrum of genes. The progesterone receptor is found in the spinal cord and nearly all cells in both males and females, and thus constitutes a useful therapeutic target for neurodegenerative diseases, e.g., ALS. A change in function of the progesterone receptor may be at the heart of many neurodegenerative conditions, including, for example, ALS, Alzheimer's disease, Parkinson's disease, Huntington's disease, and Multiple Sclerosis, each of which is described below.

Amyotrophic Lateral Sclerosis (ALS), also called Lou Gehrig's disease, is a fatal neurodegenerative disease affecting motor neurons of the cortex, brain stem and spinal cord. (Hirano, (1996) Neurology, 47 (4 Suppl. 2): S63-6). Onset of ALS occurs in the fourth or fifth decade of life (median age of onset is 57) and is fatal within two to five years after diagnosis (Williams, et al. (1991) Mayo Clin. Proc., 66: 54-82). ALS affects approximately 30,000 Americans with nearly 8,000 deaths reported in the US each year. ALS patients progressively lose all motor function—unable to walk, speak, or breathe on their own.

The cardinal feature of ALS is the loss of spinal motor neurons, which causes the muscles under their control to weaken and waste away leading to paralysis. ALS has both familial (5-10%) and sporadic forms and the familial forms have now been linked to several distinct genetic loci (Deng, et al. (1995) Hum. Mol. Genet., 4: 1113-16; Siddique, et al. (1995) Clin. Neurosci., 3: 338-47; Siddique, et al., (1997) J. Neural Transm. Suppl., 49: 219-33; Ben Hamida, et al. (1990) Brain, 113: 347-63; Yang, et al. (2001) Nat. Genet. 29: 160-65; Hadano, et al. (2001) Nat. Genet. 29: 166-73). About 15-20% of familial cases are due to mutations in the gene encoding Cu/Zn superoxide dismutase 1 (SOD1) (Siddique, et al. (1991) N. Engl. J. Med., 324: 1381-84; Rosen, et al. (1993) Nature, 362: 59-62).

Although the etiology of the disease is unknown, one theory is that neuronal cell death in ALS is the result of over-excitement of neuronal cells due to excess extracellular glutamate. Glutamate is a neurotransmitter that is released by glutaminergic neurons, and is taken up into glial cells where it is converted into glutamine by the enzyme glutamine synthetase, glutamine then re-enters the neurons and is hydrolyzed by glutaminase to form glutamate, thus replenishing the neurotransmitter pool. In a normal spinal cord and brain stem, the level of extracellular glutamate is kept at low micromolar levels in the extracellular fluid because glial cells, which function in part to support neurons, use the excitatory amino acid transporter type 2 (EAAT2) protein to absorb glutamate immediately. A deficiency in the normal EAAT2 protein in patients with ALS, was identified as being important in the pathology of the disease (See e.g., Meyer et al. (1998) J. Neurol. Neurosurg. Psychiatry, 65: 594-596; Aoki et al. (1998) Ann. Neurol. 43: 645-653; Bristol et al. (1996) Ann Neurol. 39: 676-679). One explanation for the reduced levels of EAAT2 is that EAAT2 is spliced aberrantly (Lin et al. (1998) Neuron, 20: 589-602). The aberrant splicing produces a splice variant with a deletion of 45 to 107 amino acids located in the C-terminal region of the EAAT2 protein (Meyer et al. (1998) Neureosci Lett. 241: 68-70). Due to the lack of, or defectiveness of EAAT2, extracellular glutamate accumulates, causing neurons to fire continuously. The accumulation of glutamate has a toxic effect on neuronal cells because continual firing of the neurons leads to early cell death.

Although a great deal is known about the pathology of ALS little is known about the pathogenesis of the sporadic form and about the causative properties of mutant SOD protein in familial ALS (Bruijn, et al. (1996) Neuropathol. Appl. Neurobiol., 22: 373-87; Bruijn, et al. (1998) Science 281: 1851-54). Many models have been speculated, including glutamate toxicity, hypoxia, oxidative stress, protein aggregates, neurofilament and mitochondrial dysfunction Cleveland, et al. (1995) Nature 378: 342-43; Cleveland, et al. Neurology, 47 (4 Suppl. 2): S54-61, discussion S61-2 (1996); Cleveland, (1999) Neuron, 24: 515-20; Cleveland, et al. (2001) Nat. Rev. Neurosci., 2: 806-19; Couillard-Despres, et al. (1998) Proc. Natl. Acad. Sci. USA, 95: 9626-30; Mitsumoto, (1997) Ann. Pharmacother., 31: 779-81; Skene, et al. (2001) Nat. Genet. 28: 107-8; Williamson, et al. (2000) Science, 288: 399).

Presently, there is no cure for ALS, nor is there a therapy that has been proven effective to prevent or reverse the course of the disease. Several drugs have recently been approved by the Food and Drug Administration (FDA). To date, attempts to treat ALS have involved treating neuronal degeneration with long-chain fatty alcohols which have cytoprotective effects (See U.S. Pat. No. 5,135,956); or with a salt of pyruvic acid (See U.S. Pat. No. 5,395,822); and using a glutamine synthetase to block the glutamate cascade (See U.S. Pat. No. 5,906,976). For example, Riluzole™, a glutamate release inhibitor, has been approved in the U.S. for the treatment of ALS, and appears to extend the life of at least some patients with ALS. However, some reports have indicated that even though Riluzole™ therapy can prolong survival time, it does not appear to provide an improvement of muscular strength in the patients. Therefore, the effect of Riluzole™ is limited in that the therapy does not modify the quality of life for the patient (Borras-Blasco et al. (1998) Rev. Neurol., 27: 1021-1027).

### II. SOD and SOD Mutations

The invention pertains to decreasing the SOD-1 protein (e.g., mutant SOD-1 protein) in cells by reducing or eliminating the expression of the protein with progesterone receptor modulating agents and their analogs. The SOD-1 gene is localized to chromosome 21q22.1. SOD-1 sequences are disclosed in PCT publication WO 94/19493 are oligonucleotide sequences encoding SOD-1 and generally claimed is the use of an antisense DNA homolog of a gene encoding SOD-1 in either mutant and wild-type forms in the preparation of a medicament for treating a patient with a disease. The nucleic acid sequence of human SOD-1 gene can be found at Genbank accession no. NM_{—}000454. The nucleotide sequence of human SOD-1 is also presented in SEQ ID NO: 1. The corresponding SOD-1 protein sequence is presented in SEQ ID NO: 2.

III. Compounds that Inhibit SOD Expression Through Nuclear Receptors

In one aspect, the invention pertains to using progesterone receptor modulating agents that alter gene expression or protein production of SOD, e.g., SOD-1. The progesterone receptor is a ligand activated transcription factor that binds progesterone and its analogues with high affinity and acts directly on genomic DNA to inhibit or activate the expression of a broad spectrum of genes. The progesterone receptor has been implicated in neurodegenerative disorders. In rodents and humans, the progesterone receptor gene encodes two proteins termed PRA and PRB. Both isoforms are the result of transcription of two alternative promoters and initiation of translation at two different AUG codons. Their physiological roles are different according to their structural and functional properties. PRA and PRB may activate different genes, and their ratio of expression may be important in cell fate (Richer et al. (2002) J Biol Chem 277:5209-5218 and Shyamala et al. (2000) Proc Natl Acad Sci USA, 97:3044-3049).

The progesterone receptor belongs to the nuclear receptor superfamily. Approximately 70 members of the nuclear receptor superfamily members have been identified (Moras & Gronemeyer 1998). Only some of them are ligand-binding receptors, while others belong to the subfamily of so-called orphan receptors for which specific ligands have not yet been identified or may not even exist (O'malley & Conneely 1992). The progesterone receptor can modulate gene expression directly by interacting with specific elements in the regulatory regions of target genes or indirectly by activating various growth factor signalling pathways.

The structural features of the nuclear receptor superfamily are similar. Each have four major functional regions: the N-terminal transactivation domain (TAD), a central DNA-binding domain (DBD), a C-terminal ligand-binding domain (LBD), and a hinge region connecting the DBD and LBD (Mangelsdorf et al. 1995). Two autonomous transactivation functions, a constitutively active activation function (AF-1) originating in the N-terminal and a ligand-dependent activation function (AF-2) arising in the LBD, are responsible for the transcriptional activity of nuclear receptors (Gronemeyer & Laudet 1995).

The DBD of nuclear receptors exhibits a high degree of amino acid sequence identity to other members of the subfamily. Consequently, the four receptors recognize very similar, if not identical, hormone response elements (HREs) in nuclear DNA.

Conformation changes resulting from the binding of a ligand (e.g., progesterone or estrogen) to the LBD located at the C-terminal end of the molecule are responsible for activating the ligand response. Despite the low sequence identity of as low as 20% between the LBDs of different nuclear receptor families, all nuclear receptors share a similar fold in this region. They are comprised of up to 12 helices and a small-sheet arranged in a so-called α-helical sandwich. The transactivation functions of AF-1 and AF-2 are located in the TAD and the LBD, respectively, of nuclear receptors, and the activity of them is dependent on the recruitment of coactivator molecules to form active preinitiation sites for gene transcription (Onate et al. 1998, Bevan et al. 1999). Receptors with a deletion of their LBD are constitutively active, suggesting that the AF-1 is ligand-independent. Strong AF-2 was demonstrated in LBDs of retinoic acid receptor (RAR) (Durand et al. 1994), retinoic-X receptor (RXR) (vom Baur et al. 1998), vitamin D receptor (Jimenéz et al. 1999), GR (Sheldon et al. 1999), PR (Onate et al. 1998), Peroxisome proliferatoractivated receptor (PPARγ) (Nolte et al. 1998), progesterone receptor (ER) (Tora et al. 1989), and thyroid hormone receptor (THR) (Barettino et al. 1994), but not in AR (Berrevoets et al. 1998, Bevan et al. 1999).

The transcriptional activity of nuclear receptors is affected by coregulators that influence a number of functional properties of nuclear receptor, including ligand selectivity and DNA binding capacity. Nuclear receptor coregulators participate in DNA modification of target genes, either directly through modification of histones or indirectly by the recruitment of chromatin-modifying complexes, as well as functioning in the recruitment of the basal transcriptional machinery (Heinlein & Chang 2002). Some of the better characterized coregulators are members of the p160 family, ARA70, ARA55, ARA54, ARA267-α, Smad-3, and AIB1 (Yeh et al. 1999a). ARA55 and ARA70 both allow the activation of androgen receptor by 17β-estradiol (E2), with ARA70 being the most effective coactivator for conferring androgenic activity to E2 (Miyamoto et al. 1998, Yeh et al. 1998, Fujimoto et al. 1999). Furthermore, both ARA55 and Smad-3 have been suggested to function as bridges for cross-talk between transforming growth factor-β signalling pathway and androgen/androgen receptor action (Fujimoto et al. 1999, Kang et al. 2001).

(i) Ligand Dependent Activation

Ligands, e.g., estrogen/progesterone diffuse into target cells and bind to the nuclear receptors. Ligand-binding initiates a series of events leading to the regulation of target genes by the receptor. The occupied receptor undergoes an allosteric change in its LBD, and is dissociated from heat shock proteins, such as hsp90, hsp70, and hsp56 (Roy et al. 2001), complexed, e.g., dimerized, and translocated, if it is not already present into the nucleus. Upon binding to an hormone response element (HRE) in nuclear DNA, the receptor dimer recruits coactivators such as p160 family to form an active pre-initiation complex and interacts with basal transcription machinery to inhibit or trigger the transcription of the target genes.

(ii) Ligand-Independent Activation

Nuclear receptors may also be activated by signalling pathways that originated at the cell surface. Nuclear receptors, along with other transcription factors, are regulated by reversible phosphorylation (Orti et al. 1992). Kinase-mediated signal transduction pathways could affect the activity of nuclear receptors (Burnstein & Cidlowski 1993). Certain consensus phosphorylation sites can be a substrate for the DNA-dependent protein kinase, protein kinase A, protein kinase C, mitogen-activated kinase, and casein kinase II (Blok et al. 1996).

The natural progesterone receptor modulating agent for the progesterone receptor is the progesterone ligand, but synthetic compounds, such as medroxyprogesterone acetate or levonorgestrel, norethindrone, have been made which also serve as a ligands. In one embodiment, the ligand includes, but is not limited to Amen (progestin: medroxyprogesterone), Aygestin (progestin: norethindrone), Crinone (progestin: progesterone), CombiPatch (estrogen/progestin combination: estradiol/norethindrone acetate transdermal system), Curretab (progestin: medroxyprogesterone), Cycrin (progestin: medroxyprogesterone), Depo-Provera (progestin: medroxyprogesterone), Gesterol 50 (progestin: progesterone), Gesterol LA 250 (progestin: hydroxyprogesterone), Hy/Gestrone (progestin: hydroxyprogesterone), Hylutin (progestin: hydroxyprogesterone), Megace (progestin: megestrol), Pro-Span (progestin: hydroxyprogesterone), Prodrox (progestin: hydroxyprogesterone), Prometrium (progestin: progesterone), Provera (progestin: medroxyprogesterone). Further examples of progesterone related compounds include, but are not limited to, First Generation Progestins: Norethindrone (e.g. Ortho-Novum) and Norethynodrel (Enovid); Second Generation Progestins: Norethindrone Acetate (e.g. Loestrin) and Ethynodiol Diacetate (e.g. Demulen); Third Generation Progestins: Levonorgestrel (e.g. Triphasil), d1-Norgestrel (e.g. Ovral); Fourth Generation Progestins: Desogestrel (e.g. Desogen), Gestodene, Norgestimate (e.g. Ortho-Cyclen), Pregnane Progestin, Chlormadinone acetate, Megestrol acetate, and Medroxyprogesterone acetate. In particular, levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, or (17-deacetyl) norgestimate.

In another embodiment, the ligand is a combination of ligands such as a combination of estrogen and progesterone. Examples include, but are not limited to, Premarin (estrogen: conjugated estrogens), Premelle (estrogen/progestin combination: conjugated estrogens and medroxyprogesterone), Premique (estrogen/progestin combination: conjugated estrogens and medroxyprogesterone), Premphase (estrogen/progestin combination: conjugated estrogens and medroxyprogesterone), Prempro (estrogen/progestin combination: conjugated estrogens and medroxyprogesterone), and Provelle 28 (estrogen/progestin combination: conjugated estrogens and medroxyprogesterone).

The ligand binds to the progesterone receptor to create a receptor/ligand complex. This complex binds to specific gene promoters present in nuclear DNA. Once bound to the DNA the complex modulates the production of mRNA and protein encoded by that gene. Thus, the progesterone receptor modulating agents can be FDA approved therapeutic agents that are currently being used for diseases not associated with SOD-1 function, and modified variants thereof. The progesterone receptor modulating agents can also be newly synthesized compounds that alter SOD-1 expression. The progesterone receptor modulating agents can be existing therapeutic agents known to interact with the progesterone receptor, e.g. norethindrone.

Activated progesterone receptor recruit a series of important regulatory proteins, which can serve as coactivators or corepressors, such as SRC-1, SRC-2 and SRC-3, CBP/p300 and others. These coregulatory proteins may modulate histone acetylation/deacetylation and chromatin remodeling, and may have additional effects. The progesterone receptor complex will bind a specific DNA sequence, the progesterone-responsive element, and will initiate the transcription of target genes. A complete review of the classical mechanism by which progesterone receptors are activated by their natural ligands has been extensively described elsewhere (Giangrande et al. (1999) Recent Prog Horm Res, 54:291-313). Such a complex activation sequence offers several steps in which other regulatory mechanisms of the progesterone signaling pathway can be integrated.

Progesterone receptor activation involves four sites of the progesterone receptor, that are basally phosphorylated in humans (Ser 81, Ser 162, Ser 190 and Ser 400), and exhibit a rapid twofold increase on hormone treatment. The other sites (Ser 102, Ser 294 and Ser 345) are hormone inducible, and 1-2 hours of treatment are required to reach maximal phosphorylation. Their different kinetics in response to hormone suggests that these two groups of phosphorylation sites are targets of different signaling pathways and kinases, and serve distinct functional structural roles. Phosphorylation may not serve as a regulatory on-off switch for transcriptional activity, but rather functions to either amplify or attenuate activity (Clemm et al. (2000) Mol Endocrinol; 14:52-65). Several crosstalk mechanisms involving the conventional nuclear PR pathway with different growth factors, neurotransmitters and polypeptide hormones have been described. Most studies indicate that progestins upregulate growth factor and cytokine receptors at the cell surface. They also act at the cytoplasmic level to regulate several intracellular effectors by increasing the levels and altering the subcellular compartmentalization of, for example, Stat 5, and by potentiating mitogen-activated protein kinase (MAPK) and Janus kinase activities (Lange et al. (1999) Mol Endocrinol 13:829-836).

Steroid receptors may crosstalk with each other. (Migliaccio et al. (1998) EMBO J 17:2008-2018). Migliaccio et al described an interaction between PRB, ERα and Src at the cell membrane level, which would be necessary for steroid-induced S-phase entry of cells. PR nongenomic mechanisms of action have been described in different systems (Maller et al. (2001) Proc Natl Acad Sci USA; 98:8-10).

In one aspect, the invention pertains to targeting the progesterone receptor with an progesterone receptor modulating agent, e.g., progesterone or norethindrone, to lower SOD-1 expression. Progesterone and related compounds that activate the progesterone receptor have been shown to be potent inhibitors of SOD-1 expression at the protein and the mRNA level. These compounds are thought to act through the progesterone receptor because the effects of norethindrone to decrease SOD-1 can be blocked by mifepristone, a progesterone receptor antagonist.

The Examples section shows that the expression of SOD-1 is inhibited at the level of RNA; decreased levels of RNA following norethindrone treatment suggest that this could occur by decreases in transcription or the stability of the transcript. The progesterone receptor dependent action of norethindrone, demonstrated by mifepristone antagonism, strongly suggests a direct action of the activated progesterone receptor to inhibit transcription. The decreases in SOD-1 mRNA then lead to decreased protein levels of SOD-1. Decreased levels of the SOD-1 protein reduce its accumulation in the cell and are expected to ameliorate the disease. The expression and accumulation of mutant SOD-1 is the widely accepted pathophysiological mechanism underlying familial ALS, and might also play a role in the sporadic form of the disease. The progesterone receptor is found in the spinal cord (Monks et al., (2001) Horm. Behav 40:490-496) and nearly all cells in both males and females, and thus constitutes a useful therapeutic target in all familial, and possibly sporadic ALS.

### IV. Modulation of Neurodegenerative Disorders Using Progesterone Receptor Modulating Agents

The role of the progesterone receptor in the neurodegenerative diseases such as ALS, and modulation of the pathway associated with the progesterone receptor has not been the target of a clinical investigation in ALS or other neurodegenerative disease. The data shown in the Examples section indicate that the progesterone receptor plays a role in decreasing the expression of SOD-1.

The SOD1 G93A (high copy) mouse model for ALS is a suitable mouse that carries 23 copies of the human G93A SOD mutation and is driven by the endogenous promoter. Survival in the mouse is copy dependent. The high copy G93A has a median survival of around 128 days. High molecular weight complexes of mutant SOD protein are seen in the spinal cord beginning around day 30. At day 60 reactive astrocytosis (GFAP reactive) are observed; activated microglia are observed from day 90 onwards. Studies by Gurney et al. showed that at day 90 reactive astrocytosis loses statistical significance while microglial activation is significantly elevated and continues to be elevated through the end stage of the disease (See Gurney, et al. (1996) Ann. Neurol., 39: 147-5739).

Many drugs that have shown efficacy in this model have moved forward into human clinical trials. Experience with riluzole, the only approved drug in the treatment of ALS, indicates that the mouse ALS model is a good predictor of clinical efficacy. Other drugs such as Creatine, Celebrex, Co-enzyme Q10, and Minocycline are under clinical evaluation based on studies in this model.

### V. Delivery of the Progesterone Receptor Modulating Pharmacological Agents

The pharmacological agent of the present invention can be incorporated into pharmaceutical compositions suitable for administration to a subject. Typically, the pharmaceutical composition comprises a progesterone receptor modulating pharmacological agent, e.g., norethindrone and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. The preferred mode of administration is parenteral (e.g., intravenous, subcutaneous, intraperitoneal, intramuscular). In a preferred embodiment, the pharmacological agent is administered by an intraperitoneal injection.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, (see, for example, Langer, Science 249, 1527 (1990) and Hanes, Advanced Drug Delivery Reviews 28, 97-119 (1997). The agents of this invention can also be administered in the form of a depot injection or implant preparation which can be formulated in such a manner as to permit a sustained or pulsatile release of the active ingredient. The depot injection or implant preparation can, for example, comprise one or more of the compounds of the present invention, or comprise a combination of different agents (e.g., pyrimethamine and norethindrone).

The pharmaceutical compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., the pharmacological agent) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The progesterone receptor modulating pharmacological agent can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. (See, e.g., Sustained and Controlled Release Drug Delivery Systems, J. R. Robinson, ed., Marcel Dekker, Inc., New York, 1978; U.S. Pat. Nos. 6,333,051 to Kabanov et al., and 6,387,406 to Kabanov et al.).

In certain embodiments, a progesterone receptor modulating pharmacological agent may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In certain embodiments, a progesterone receptor modulating pharmacological agent can be administered in a liquid form. The pharmacological agent should be soluble in a variety of solvents, such as for example, methanol, ethanol, and isopropanol. A variety of methods are known in the art to improve the solubility of the pharmacological agent in water and other aqueous solutions. For example, U.S. Pat. No. 6,008,192 to Al-Razzak et al. teaches a hydrophilic binary system comprising a hydrophilic phase and a surfactant, or mixture of surfactants, for improving the administration of compounds.

Supplementary active compounds can also be incorporated into the compositions. In certain embodiments, a progesterone receptor modulating pharmacological agent can be coformulated with and/or coadministered with one or more additional therapeutic agents that are useful for improving the pharmacokinetics of the pharmacological agent. A variety of methods are known in the art to improve the pharmacokinetics of the pharmacological agent of the present invention. (See e.g., U.S. Pat. No. 6,037,157 to Norbeck et al.).

Other methods of improving the pharmacokinetics of the pharmacological agent have been disclosed, for example, in U.S. Pat. Nos. 6,342,250 to Masters, 6,333,051 to Kabanov et al., 6,395,300 to Straub et al., 6,387,406 to Kabanov et al., and 6,299,900 to Reed et al. Masters discloses a drug delivery device and method for the controlled release of pharmacologically active agents. The drug delivery device disclosed by Masters is a film comprising one or more biodegradable polymeric materials, one or more biocompatible solvents, and one or more pharmacologically active agents dispersed uniformed throughout the film. In U.S. Pat. No. 6,333,051, Kabanov et al. disclose a copolymer networking having at least one cross-linked polyamine polymer fragment, at least one nonionic water-soluble polymer fragment, and at least one suitable biological agent, including a pharmacological agent. According to the teachings of this patent, this network, referred to as a nanogel network, improves the therapeutic effect of the pharmacological agent by decreasing side effects and increasing therapeutic action. In another patent, U.S. Pat. No. 6,387,406, Kabanov et al. also disclose another composition for improving the oral delivery of numerous pharmacological agents.

Other methods for improving the delivery and administration of the pharmacological agent include means for improving the ability of the pharmacological agent to cross membranes, and in particular, to cross the blood-brain barrier. In one embodiment, the pharmacological agent can be modified to improve its ability to cross the blood-brain barrier, and in an alternative embodiment, the pharmacological agent can be co-administered with an additional agent, such as for example, an anti-fungal compound, that improves the ability of the pharmacological agent to cross the blood-brain barrier. Alternatively, precise delivery of the pharmacological agent into specific sites of the brain, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for pharmacological agent microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The pharmacological agent can be delivered to regions, such as the cells of the spinal cord, brainstem, or brain that are associated with the disease or disorder. For example, target regions can include the medulla, pons, and midbrain, cerebellum, diencephalon (e.g., thalamus, hypothalamus), telencephalon (e.g., corpus stratium, cerebral cortex, or within the cortex, the occipital, temporal, parietal or frontal lobes), or combinations, thereof.

Progesterone receptor modulating pharmacological agents can be used alone or in combination to treat neurodegenerative disorders. For example, the pharmacological agent can be used in conjunction with other existing progesterone receptor modulators, for example, to produce a synergistic effect. Likewise, the pharmacological agent can be used alone or in combination with an additional agent, e.g., an agent which imparts a beneficial attribute to the therapeutic composition, e.g., an agent which effects the viscosity of the composition. The combination can also include more than one additional agent, e.g., two or three additional agents if the combination is such that the formed composition can perform its intended function. In some embodiments, the invention includes administrating at least one progesterone related compound, such as norethindrone, together with for example, with at least one pyrimethamine compound or functional analog thereof, or with at least one estrogen related compound, such as estradiol. For descriptions of these compounds and administration, see co-pending applications entitled “Modulation of Neurodegenerative Diseases through the Progesterone Receptor” and “Modulation of Neurodegenerative Diseases” filed Mar. 1, 2006.

The compounds of the present invention can be conjugated with pharmaceutically acceptable acid salts to facilitate their long storage and dosing as aqueous solutions. For example, the salt can be derived from a pharmaceutically acceptable acid (e.g., HCl) with or without the use of a pharmaceutically acceptable carrier (e.g., water). Such salts can be derived from either inorganic or organic acids, including for example hydrochloric, hydrobromic, acetic, citric, fumaric, maleic, benzenesulfonic, and ascorbic acids. The pharmaceutical compositions obtained by the combination of the carrier and the salt will generally be used in a dosage necessary to elicit the desired biological effect. This includes its use in a therapeutically effective amount or in a lesser amount when used in combination with other biologically active agents.

The pharmaceutical compositions of the invention may include a “therapeutically effective amount” or a “prophylactically effective amount” of a pharmacological agent of the invention. A “therapeutically effective amount” refers to an amount effective, at dosages and for periods of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a pharmacological agent (e.g., progesterone or norethindrone) is between 0.5 mg/day to about 20 mg/day administered to a subject, or group of subjects, preferably about 0.100 mg/day to about 15 mg/day, more preferably about 0.100 mg/day to about 12 mg/day, and most preferably about 0.3 mg/day to 4 mg/day. Preferably, administration of a therapeutically effective amount of pharmacological agent (e.g., progesterone or norethindrone), results in a concentration of pharmacological agent in the bloodstream in the range of 1 nanomolar (nM) to 100 millimolar (mM) concentration. For example, a concentration range of about 10 nM to about 10 mM, about, 1 nM to about 1 mM, about 1 nM to about 100 micromolar (μM), about 1 μM to about 500 μM, about 1 μM to about 200 μM, or about 10 μM to about 50 μM. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

### Materials and Methods

### (i) Cell Culture

The human cervical carcinoma derived HeLa cell line (ATCC) was found to express SOD-1 protein and mRNA and was used as the model system to identify compounds that inhibit SOD-1 expression. Briefly, cells were maintained in Dulbecco's Minimal Essential Medium, with high glucose, supplemented with glutamine, 4 mM, certified fetal bovine serum, 10%, and penicillin, streptomycin, and nystatin (all from Invitrogen). Incubation conditions were 37 degrees and 99% relative humidity, with CO₂ at 5%. Cultures were passaged when they reached 90% confluence. For pharmacological experiments, cells were plated into sterile tissue culture treated 96 well plates at a density of 3,500 cells/well in 150 μl medium.

### (ii) Drugs:

All compounds were dissolved in 100% DMSO, at a stock concentration of 10 mM. Drugs were obtained from Microsource Discovery or from Sigma Aldrich.

(iii) Experimental Protocol:

After plating and 6 hours for attachment, drugs were added to the medium in a concentration of 10 μM. Following 72 hours of incubation with the drugs, the cells were photographed at 100× using an inverted microscope and digital camera, so that cytotoxicity could be evaluated. After photodocumentation, the medium was removed and the cells were washed once with phosphate buffered saline, and then 50 μl molecular biology grade water containing a protease inhibitor cocktail was added. After 10 min incubation, the plates were placed in −80 degrees to induce complete lysis. Plates were then thawed and 25 μl was transferred from each well into a maxsorp ELISA plate coated with anti-human SOD-1 antibody, which contained 75 μl phosphate buffered saline. A second antibody pair (a polyclonal anti-SOD-1/HRP conjugated goat anti-rabbit) was then added to the well, and incubation was conducted for 1 hour at room temperature. At the conclusion of the incubation, the plate was washed three times (wash buffer from KPL Inc.) and Sure Blue Reserve HRP Substrate was added. Following a 5-10 min incubation, the reaction (which had turned blue to varying degrees) was stopped by the addition of a stop reagent (KPL). The plate was then shaken gently for 5 seconds and the absorbance at 450 nm read on a Tecan Plate reader. Absorbance from each sample were compared to standard curve of purified recombinant human SOD-1 assayed on the same ELISA plate, and SOD-1 immunoreactivity (ng/ml) was estimated by comparison with the standard curve.

### (iv) Bradford Protein Assay:

To determine if decrements found in the SOD-1 assay were simply the result of cytotoxic effects of the drug treatment, total protein was determined for each well. While the ELISA incubation was ongoing, 10 μl of the remaining lysate was removed from each well and placed into another empty plate, and BioRad Bradford reagent (100 μl) was added to the protein. After a 15 min incubation at room temperature the plate was shaken gently for 5 seconds and the absorbance was read at 595 nm in a Tecan Sunrise plate reader. Protein concentrations in each well were thus determined by comparison with protein standards that were run on the same plate.

### (v) Quantitative RT-PCR:

HeLa cells at 3500 cells/well in a 96 well plate were treated with norethindrone for 72 h as above and then cells were lysed and total RNA extracted using the Gentra RNA extraction protocol and reagents. The purified RNA was then used as the template in a reverse transcription reaction using Superscript III MMLV Transcriptase primed with oligoDT. A PCR reaction was performed on the resultant cDNA to amplify the cDNA corresponding to human SOD-1, human TATA-box binding protein, and human Beta-2 microglobulin. The PCR reactions were run in separate tubes for 20, 25, and 30 cycles and the amplicons were then run on a 2% agarose gel containing ethidium bromide. The fluorescence emitted by the ethidium bromide stained bands following stimulation by a UV light source was captured using a digital camera. The digitized images were analyzed using ImageJ (NIH) and the bands for SOD-1 were compared with the bands for TATA-box binding protein and Beta2 Microglobulin (these housekeeping genes were unaffected by the drugs) while in the linear range of cycles, 25 cycles under these conditions, for increases or decreases relative to controls.

### (vi) GeneChip Experiments:

Total cellular mRNA was prepared from HeLa cells with or without treatment using a Qiagen RNA mini kit followed by oligotex mRNA mini kit. Double-stranded cDNAs were synthesized from 2 μg total mRNA using the Superscript Choice System for cDNA synthesis (Invitrogen) with the T7-(dT)24 primer following the manufacturer's recommendations. cDNAs were cleaned up by phase lock gel (PLG) phenol/chloroform extraction and concentrated by ethanol precipitation. Biotin-labeled cRNA was synthesized from cDNA by in vitro transcription using the Bioarray HighYield RNA transcript Labeling Kit (Affymetrix) following vendor's recommendation. In vitro transcription products were cleaned up using RNeasy spin columns (Qiagen) and fragmented by metal-induced hydrolysis in fragmentation buffer (40 mM Tris-acetate, pH 8.1, 100 mM KOAc, 30 mM MgOAc). Fragmented cRNA was then subjected to Affymetrix GeneChip sets in hybridization buffer (100 mM MES, 1M NaCl, 20 mM EDTA, 0.01% Tween-20). GeneChip images were analyzed with Affymetrix Microarray Suite V5.0 and Affymetrix Data Mining Tool V3.0. Signal intensities of all probe sets were scaled to a target value of 150. Results of Detection Call, Change Call and Signal Log Ratio were obtained by applying the default parameters to statistical algorithms for both absolute and comparison analyses.

(vii) Western Blotting.

Animals were overdosed with sodium pentobarbital (250 mg/kg, i.p.). Spinal cords were dissected and homogenized in 20 mM Tris-HCl, pH 7.5, 2 mM DTT, 0.1 mg of leupeptin, 1 mM EDTA, and 1 mM EGTA. The homogenate was then centrifuged at 14,000×g to pellet debris. Protein concentration was measured using the BCA protein assay (Pierce, Rockford, Ill.). Protein (25 μg) from each sample was run on a 4-20% Tris-glycine gel (Invitrogen, San Diego, Calif.). After transfer, membranes were washed in PBS, followed by overnight incubation in blocking buffer (0.2% I-block (Applied Biosystems, Foster City, Calif.), PBS, and 0.1% Tween 20). The membrane was then probed with a polyclonal rabbit anti-bovine SOD1 (Sigma) antibody at 1:4000 dilution. After several washes, membranes were incubated with an alkaline phosphatase-conjugated secondary antibody (1:5000 in blocking buffer), and the immunoreactive signals were visualized using an enhanced chemiluminescent reagent, CDP Star (Western Star kit; Tropix). After exposure, films were scanned and then imported into NIH Image for quantitation of band density.

### Example 2

### Testing the Effects of on a Progesterone Receptor

This example describes how to examine the in vitro effects of a progesterone receptor drug, e.g., norethindrone, on SOD-1 activity. The human cervical carcinoma derived HeLa cell line (ATCC) were cultured in Dulbecco's Minimal Essential Medium, with high glucose, supplemented with glutamine, 4 mM, certified fetal bovine serum, 10%, and penicillin, streptomycin, and nystatin (all from Invitrogen). Incubation conditions were 37° C. and 99% relative humidity, with CO₂ at 5%. Cultures were passaged when they reached 90% confluence. For pharmacological experiments, cells were plated into sterile tissue culture treated 96 well plates at a density of 3,500 cells/well in 150 μl medium.

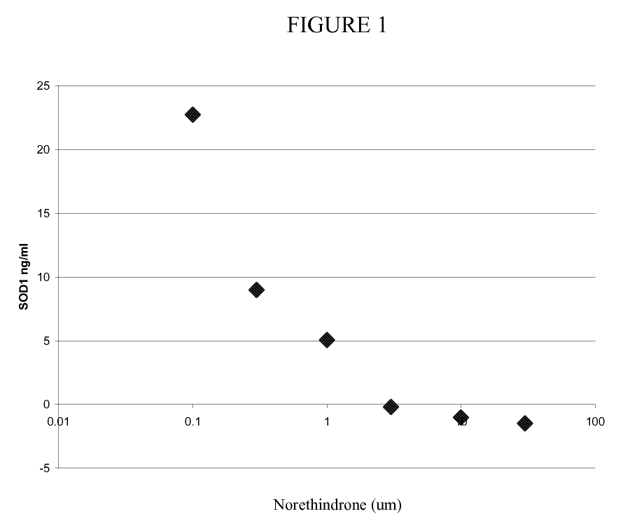

Following 72 hours of incubation with the drugs, the cells were photographed and processed as described in Example 1 (iii). The total protein of the lysates was determined by Bradford assay as described in Example 1 (iv). The results of this study are shown in FIG. 1. These results show that norethindrone added to culture medium of HeLa cells 72 hours before harvest significantly reduced the levels of SOD-1 protein, while total protein levels were unaffected. This reduction was dose related and maximal by 3 μM, with an IC₅₀ of 2 μM.

Alpha-synuclein has been implicated in neurodegenerative disorders characterized by Lewy body inclusions such as Parkinson's disease (PD) and dementia with Lewy bodies. Lewy body-like inclusions have also been observed in spinal neurons of patients with amyotrophic lateral sclerosis (ALS) and reports suggest possible alpha-synuclein abnormalities in ALS patients alpha-Synuclein is a ubiquitous protein that shares significant physical and functional homology to the protein chaperone, 14-3—3, and is particularly abundant in the brain (Ostrerova N. et al., J. Neurosci., 19:5782 (1990)). An increased rate of alpha-synuclein aggregation might contribute to the mechanisms of neurodegeneration in Lewy body diseases. Studies on transgenic animals also suggest that aggregation of alpha-synuclein is harmful to neurons. It was reported that dopaminergic dysfunction occurred in transgenic mice expressing wild type human alpha-synuclein (Masliah, E., et al., Science, 287:1265-1269 (2000)) and that Drosophila over-expressing alpha-synuclein exhibited dopaminergic dysfunction and dopaminergic neuronal death associated with development of alpha-synuclein aggregates (Feany, M B, et al., Nature 404:394-8 (2000)). Evidence suggests that neurons with dopamine develop alpha-synuclein aggregates and degenerate as these aggregates development.

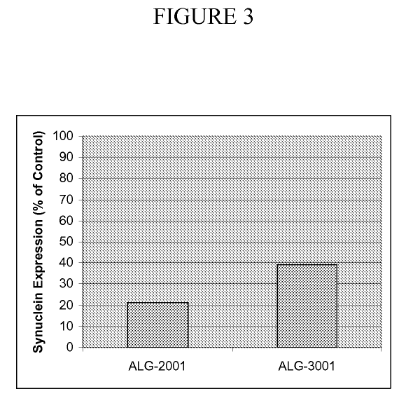

The results of the Genechip analysis shown in FIG. 3 illustrates that pyrimethamine (ALG-2001) (3 μM) and norethindrone (ALG-3001) (3 μM) substantially decreased mRNA for alpha synuclein in HeLa cells following 4 days of treatment. Thus, the compounds of the present invention can slow neurodegeneration in Lewy body diseases. This illustrates a role for the compounds of the present invention in slowing the progression or ameliorating the effects of ALS and PD.

### Example 3

### Testing the Effects of Compounds In Vivo: SOD-93A Murine Model

The effects of the progesterone receptor modulating agent (e.g., norethindrone), and analogs thereof described in Examples 2 were tested in vivo in the SOD-93A murine model for ALS, and a reduction in SOD-1 levels was measured. The inhibition of RNA expression was monitored by isolated blood samples from a mouse pre- and post introduction of the compound using standard RT-PCR techniques. The expression of the SOD-1 protein was determined using Western blot techniques with an anti-SOD-1 antibody from Sigma.

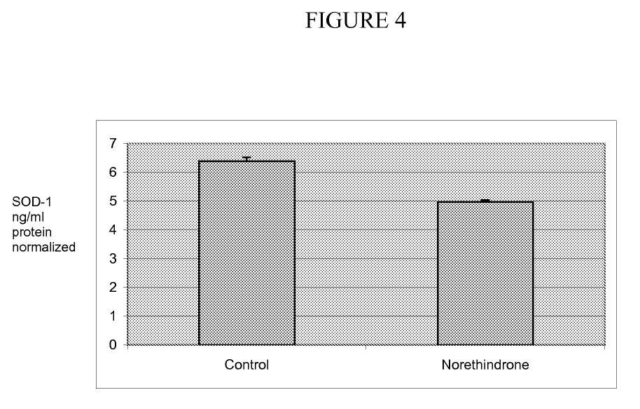

Chronic administration of norethindrone (100 mg/kg/d) for 14 days significantly (p=0.000351, n=5/group) decreased spinal SOD-1 in G93A male mice as shown in FIG. 4. Norethindrone was administered orally for 14 days. Spinal cords were harvested and analyzed by ELISA and Western blot analysis as described above. The control group was given vehicle (saline) only.

The in vivo effects can also be determined by monitoring the breathing of a subject by measuring the forced vital capacity (FVC) using a Renaissance Puritan Bennett Spirometer. The maximum inspiratory force (MIF) can also be measured using a hand held manometer.

### Example 4

### Neurological Scoring

The effects of the progesterone receptor modulating agent can also be determined by a neurological score recorded on a 4-point scale:
0=Normal reflex on the hind limbs (animal will splay its hind limbs when lifted by its tail)
1=Abnormal reflex (Lack of splaying of hind limbs when animal is lifted by the tail).
2=Abnormal reflex and visible evidence of paralysis
3=Lack of reflex and total paralysis of hind limbs.
4=Inability to right themselves when placed on the sides in 30 seconds or found dead. The animals are sacrificed at this stage if alive.

Statistical analysis on the neurological score, body weight and survival can be performed by utilizing ANOVA, Kaplan Meier, t-test, Cox's proportional hazards regression model log-logistic and panametric methods and mixed linear model methods. All statistical analysis was performed using standard procedures known in the art.

1. A method for reducing the production of a superoxide dismutase (SOD) protein in a cell of a subject with a neurodegenerative disorder comprising, delivering a progesterone receptor modulating pharmacological agent to the cell, such that the agent interacts with a progesterone receptor and inhibits transcription of a gene encoding the SOD protein, wherein the agent is selected from the group consisting of progesterone and analogs thereof, norethindrone and analogs thereof, or combinations thereof.

2. The method of claim 1, wherein the cell is selected from the group consisting of a cell in a brain, a cell in a spinal cord, a cell in a meningial tissue and, a cell in a muscle.

3. The method of claim 1, wherein the cell is a neural cell in a subject with Amyotrophic Lateral Sclerosis (ALS).

4. The method of claim 1, wherein the SOD protein is the SOD-1 protein.

5. (canceled)

6. (canceled)

7. The method of claim 1, wherein the progesterone receptor modulating pharmacological agent is selected from the group consisting of levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, or (17-deacetyl) norgestimate, and analogs thereof.

8. The method of claim 1, wherein the inhibition of transcription of the gene comprises monitoring expression levels of the SOD protein.

9. The method of claim 1, wherein the inhibition of transcription of the gene comprises monitoring the levels of a nucleic acid molecule that encodes the SOD protein.

10. The method of claim 9, wherein the nucleic acid molecule is selected from the group consisting of ribonucleic acid or deoxynucleic acid.

11. A method for preventing the development of symptoms, or ameliorating the symptoms or progression of familial amyotrophic lateral sclerosis (ALS) in a subject comprising, administering a prophylactically therapeutically effective amount of a progesterone receptor modulating pharmacological agent to the subject, wherein the agent interacts with an progesterone receptor and inhibits transcription of a gene encoding a SOD-1 protein, wherein the agent is selected from the group consisting of progesterone and analogs thereof, norethindrone and analogs thereof, or combinations thereof.

12. (canceled)

13. (canceled)

14. The method of claim 11, wherein the progesterone receptor modulating pharmacological agent is selected from the group consisting of levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, or (17-deacetyl) norgestimate, and analogs thereof.

15. The method of claim 11, further comprising monitoring the amelioration of ALS by monitoring survival prolongation of the subject.

16. The method of claim 15, wherein the step of monitoring the amelioration of ALS comprises monitoring a neurological score of the subject.

17. The method of claim 15, wherein the step of monitoring the amelioration of ALS comprises monitoring expression levels of the SOD-1 protein.

18. The method of claim 15, wherein the step of monitoring the amelioration of ALS comprises monitoring the levels of a nucleic acid molecule that encodes SOD-1.

19. The method of claim 18, wherein the nucleic acid molecule is selected from the group consisting of ribonucleic acid or deoxynucleic acid.


SEQUENCE LISTING (2 sequences)

SEQ ID NO: 1 - DNA, 2288 bp, Homo sapiens
gtaccctgtttacatcattttgccattttcgcgtactgcaaccggcgggccacgccgtgaaaagaaggttgttttctccacagtttcggggttctggacgtttcccggctgcggggcggggggagtctccggcgcacgcggccccttggcccgccccagtcattcccggccactcgcgacccgaggctgccgcagggggcgggctgagcgcgtgcgaggccattggtttggggccagagtgggcgaggcgcggaggtctggcctataaagtagtcgcggagacggggtgctggtttgcgtcgtagtctcctgcaggtctggggtttccgttgcagtcctcggaaccaggacctcggcgtggcctagcgagttatggcgacgaaggccgtgtgcgtgctgaagggcgacggcccagtgcagggcatcatcaatttcgagcagaaggcaagggctgggaccgggaggcttgtgttgcgaggccgctcccgacccgctcgtccccccgcgaccctttgcatggacgggtcgcccgccagggctagagcagttaagcagcttgctggaggttcactggctagaaagtggtcagcctgggattgcatggacggatttttccactcccaagtctggctgctttttacttcactgtgaggggtaaaggtaaatcagctgttttctttgttcagaaactctctccaactttgcacttttcttaaaggaaagtaatggaccagtgaaggtgtggggaagcattaaaggactgactgaaggcctgcatggattccatgttcatgagtttggagataatacagcaggtgggtcataatttagcttttttttcttcttcttataaataggctgtaccagtgcaggtcctcactttaatcctctatccagaaaacacggtgggccaaaggatgaagagaggtaacaagatgcttaactcttgtaatcaatggcgatacgtttctggagttcatatggtatactacttgtaaatatgtgcctaagataattccgtgtttcccccacctttgcttttgaacttgctgactcatgtgaaaccctgctcccaaatgctggaatgcttttacttcctgggcttaaaggaattgacaaatgggcacttaaaacgatttggttttgtagcatttgattgaatatagaactaatacaagtgccaaaggggaactaatacaggaaatgttcatgaacagtactgtcaaccactagcaaaatcaatcatcatttgatgcttttcatataggcatgttggagacttgggcaatgtgactgctgacaaagatggtgtggccgatgtgtctattgaagattctgtgatctcactctcaggagaccattgcatcattggccgcacactggtggtaagttttcataaaggatatgcataaaacttcttctaacagtacagtcatgtatctttcactttgattgttagtcgcgaattctaagatccagataaactgtgtttctgcttttaaactactaaatattagtatatctctctactaggattaatgttatttttctaatattatgaggttcttaaacatcttttgggtattgttgggaggaggtagtgattacttgacagcccaaagttatcttcttaaaattttttacaggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaattagctctgatacttatttatataaacagcttcagtggaacagatttagtaatactaacagtgatagcattttattttgaaagtgttttgagaccatcaaaatgcatactttaaaacagcaggtcttttagctaaaactaacacaactctgcttagacaaataggctgtcctttgaagctt

SEQ ID NO: 2 - protein, 153 aa, Homo sapiens
ATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ